(19) United States
(12) Patent Application Publication
(10) Pub. No.: US 2004/0010209 A1
(43) Pub. Date: Jan. 15, 2004

(54) Device and method for measuring the diameter of an air passageway

(75) Inventor: William A. Sirokman, Kirkland, WA (US)

Correspondence Address: KNOBBE MARTENS OLSON & BEAR LLP, 2040 MAIN STREET, FOURTEENTH FLOOR, IRVINE, CA, 92614, US

(73) Assignee: Spiration, Inc.

(21) Appl. No.: 10/196,513

(22) Filed: Jul. 15, 2002

(51) Int. Cl. A61B 5/103

(52) U.S. Cl. 600/587

(57) ABSTRACT

A device and method measuring inside diameter of a body lumen. A method includes placing a balloon inside a lumen, the balloon expandable to a transverse dimension in response to fluid ejected from the fluid dispenser, and having a known relationship between volume and the expandable transverse dimension, expanding the balloon until the expandable transverse dimension is adjacent to an interior wall, and determining the lumen diameter in response to the volume of the expanded balloon. A device includes a catheter having an inflation lumen, a fluid dispenser in fluid communication with the inflation lumen and operable to eject a measurable volume of fluid, and a balloon member in fluid communication with the inflation lumen and expandable to a transverse dimension adjacent to opposing portions of an interior wall in response to fluid ejected from the fluid dispenser, and having a known relationship between volume and the expandable transverse dimension.

### BACKGROUND

The present invention is generally directed to a device, system, and method for measuring the inside diameter of a body lumen and, more particularly, of air passageways. The present invention is more particularly directed toward measuring a diameter of an air passageway by transorally inserting a balloon having a known volume-to-diameter relationship in the air passageway, expanding the balloon until it contacts the air passageway with fluid, and determining the diameter as a function of the fluid volume.

Several emerging technologies employ devices placed in the air passageways to diagnose and treat conditions of the lung, conditions of organs and body structures that are in proximity to the lungs, and of conditions that are systemic. For example, a treatment for Chronic Obstructive Pulmonary Disease (COPD) involves placing obstructing devices in selected air passageways to collapse lung portions distal of the obstructing devices. The devices are typically placed in air passageways between approximately 4 and 10 mm in diameter.

The performance of intra-bronchial devices may be enhanced by sizing the device to fit the air passageway. However, no method or device presently exists for determining the inside diameter of an air passageway. There is a need in the art for quickly and economically measuring the inside diameter of an air passageway to assist in selecting the size of an obstructing device.

In view of the foregoing, there is a need in the art for a new and improved apparatus and method for measuring the inside diameter of air passageways.

### SUMMARY

An aspect of the invention provides a device for measuring an inside diameter of an air passageway. The device includes a flexible catheter having an inflation lumen, a fluid dispenser in fluid communication with the inflation lumen and operable to eject a measurable volume of fluid, and a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension. The balloon member may be dimensioned for transoral placement into the air passageway. The catheter may be configured to be steerable within bronchi. The balloon member may include a non-complaint material, and may be arranged to expand into contact with the air passageway wall. The transverse dimension balloon may be arranged to expand to a dimension of between 3 mm and 12 mm. The balloon may have a deflated configuration for placement in the air passageway and an inflated configuration for measuring the diameter of the air passageway. The balloon may be arranged to transition from the inflated configuration to the deflated configuration while in the air passageway, and then to transition from the deflated configuration to a re-inflated configuration for measuring the diameter of another air passageway. The fluid dispenser may include a syringe, and may further include gradations corresponding to air passageway diameters. The catheter may have a distal end, and the balloon may be carried on the catheter proximate to the distal end of the catheter.

Another embodiment of the invention provides an assembly for use in measuring the diameter of an air passageway. The assembly includes a flexible catheter having an inflation lumen, the inflation lumen being arranged for fluid coupling with a fluid dispenser operable to eject a measurable volume of fluid, and a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension. The balloon may be carried on the catheter. The catheter may have a distal end, and the balloon may be carried proximate to the distal end of the catheter. The balloon member may be arranged to expand into contact with the air passageway wall.

A further embodiment of the invention provides a device for measuring an inside diameter of an air passageway. The device includes a flexible catheter having an inflation lumen, a fluid dispenser in fluid communication with the inflation lumen and operable to eject a measurable volume of fluid, a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension, and a port in fluid communication with the inflation lumen allowing sensing of pressure in the inflation lumen. The device may further include a pressure indicator, and the pressure indicator may be coupled to the port. The pressure indicator may be arranged to measure luminal pressure between zero mmHg and 700 mmHg. The pressure indicator may include a pressure sensor that generates a sensor signal and an indicator operable to indicate luminal pressure in response to the sensor signal. The fluid dispenser may include a syringe or a syringe pump. The device may further include a pressure sensor coupled to the port, and a controller coupled to the fluid dispenser and the pressure sensor. The controller being operable to control fluid ejection, determine volume of fluid ejected before a predetermined pressure occurs in the inflation lumen, determine air passageway diameter in response to volume of fluid ejected, and display determined air passageway diameter. The balloon member may be arranged for transoral placement into the air passageway.

Still another embodiment of the invention provides a device for measuring an inside diameter of an air passageway. The device includes a flexible catheter having an inflation lumen, a fluid dispenser in fluid communication with the inflation lumen and operable to eject a measurable volume of fluid, and a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension. The device also includes a pressure indicator coupled to the inflation lumen for indicating pressure in the inflation lumen. The balloon member may be arranged to expand into contact with the air passageway wall.

In yet another embodiment of the invention, a device for measuring an inside diameter of a body lumen is provided. The device includes a flexible catheter having an inflation conduit, a fluid dispenser in fluid communication with the inflation conduit and operable to eject a measurable volume of fluid, and a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension. An additional embodiment of the invention provides a method of measuring an air passageway diameter. The method includes the steps of placing a balloon in the air passageway, the balloon having a known relationship between volume and an expandable transverse dimension, expanding the balloon until the expandable transverse dimension contacts opposing portions of an inner periphery of the air passageway, and determining the air passageway diameter in response to the volume of the expanded balloon. The method may further include the step of detecting contact with the inner periphery of the air passageway. The step of detecting contact may include the further step of visually establishing contact. The step of detecting contact may include the further step of sensing a predetermined pressure in the balloon. The method may further include the step of placing a fluid dispenser in fluid communication with the balloon, the fluid dispenser being operable to inject a measurable volume of fluid into the balloon, and the step of expanding the balloon includes the further step of injecting a measurable volume of fluid into the balloon. The fluid dispenser may include a syringe. The fluid dispenser may include gradations related to air passageway diameter, and the step of determining air passageway diameter may include the further step of observing the gradations. The step of placing a balloon in the air passageway may include the further step of transorally placing the balloon in the air passageway.

Another aspect of the invention provides a device for measuring an inside diameter of an air passageway. The device includes means for placing a balloon in the air passageway, the balloon expandable in a transverse dimension and a known relationship between volume and expandable transverse dimension, means for expanding the balloon until the expandable transverse dimension contacts opposing portions of an inner periphery of the air passageway, and means for determining the air passageway diameter in response to the volume of the expanded balloon.

These and various other features as well as advantages which characterize the present invention will be apparent from reading the following detailed description and a review of the associated drawings.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 is a sectional view of a healthy respiratory system;

FIG. 2 is a perspective view of the bronchi emphasizing the upper right lung lobe;

FIG. 3 illustrates a respiratory system suffering from COPD;

FIG. 4 illustrates an air passageway inside diameter measuring device in accordance with the present invention;

FIG. 5 illustrates a step in measuring an inside diameter of an air passageway at a measuring location with the measuring device of FIG. 4 in accordance with an aspect of the invention;

FIG. 6 illustrates another step in measuring an inside diameter of an air passageway at a measuring location with the measuring device of FIG. 4 in accordance with an aspect of the invention;

FIG. 7 illustrates another embodiment of an air passageway inside diameter measuring device in accordance with the present invention; and

FIG. 8 illustrates still another embodiment of an air passageway inside diameter measuring device in accordance with the present invention.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined only by the appended claims.

Throughout the specification and claims, the following terms take the meanings explicitly associated herein unless the context dictates otherwise. The meaning of “a”, “an”, and “the” include plural references. The meaning of “in” includes “in” and “on.” Referring to the drawings, like numbers indicate like parts throughout the views. The term “coupled” means either a direct connection between the things that are coupled, or an indirect connection through one or more intermediary devices. Additionally, a reference to the singular includes a reference to the plural unless otherwise stated or inconsistent with the disclosure herein.

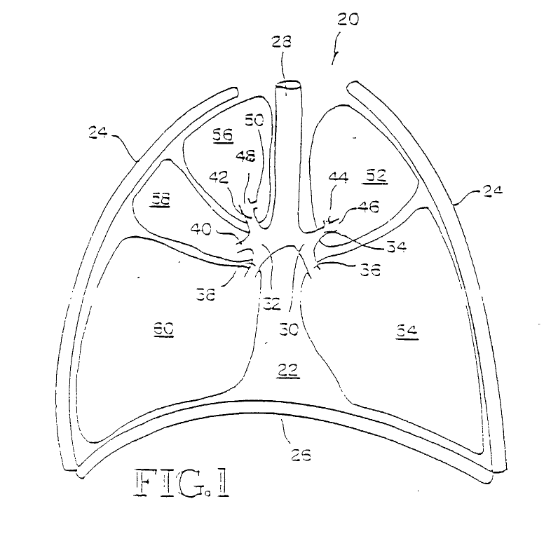

FIG. 1 is a sectional view of a healthy respiratory system. The respiratory system 20 resides within the thorax 22 that occupies a space defined by the chest wall 24 and the diaphragm 26.

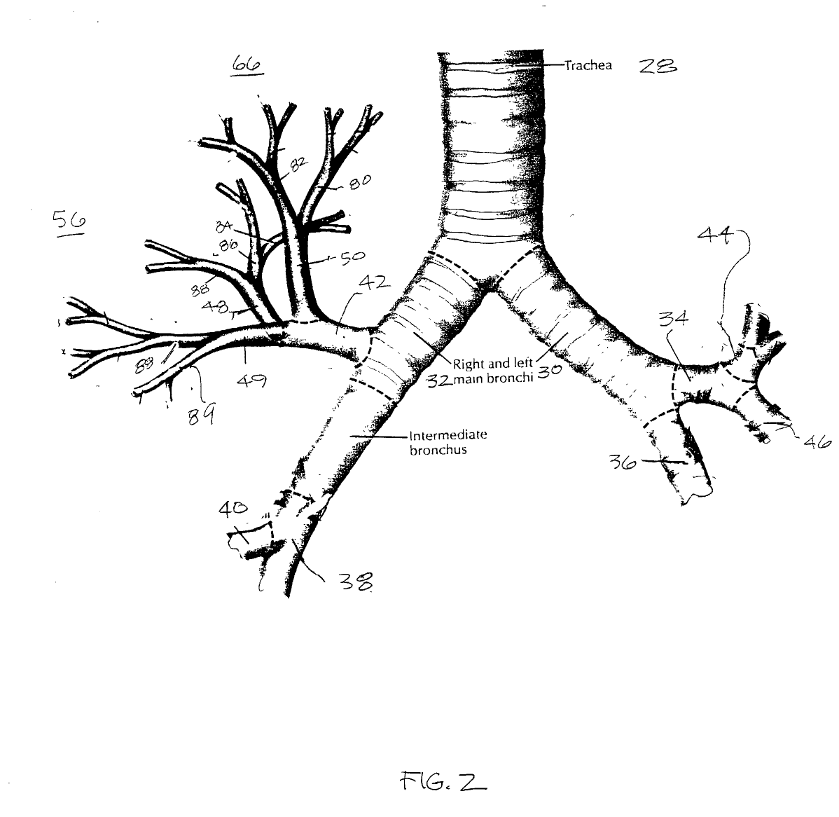

The respiratory system 20 includes trachea 28; left mainstem bronchus 30 and right mainstem bronchus 32 (primary, or first generation); and lobar bronchial branches 34, 36, 38, 40, and 42 (second generation). FIG. 1 also illustrates segmental branches 44, 46, 48, and 50 (third generation). Additional sub-branches are illustrated in FIG. 2. The respiratory system 20 further includes left lung lobes 52 and 54 and right lung lobes 56, 58, and 60. Each bronchial branch and sub-branch communicates with a different portion of a lung lobe, either the entire lung lobe or a portion thereof. As used herein, the term “air passageway” is meant to denote either a bronchi or bronchioli, and typically means a bronchial branch of any generation.

A characteristic of a healthy respiratory system is the arched or inwardly arcuate diaphragm 26. As the individual inhales, the diaphragm 26 straightens to increase the volume of the thorax 22. This causes a negative pressure within the thorax. The negative pressure within the thorax in turn causes the lung lobes to fill with air. When the individual exhales, the diaphragm returns to its original arched condition to decrease the volume of the thorax. The decreased volume of the thorax causes a positive pressure within the thorax, which in turn causes exhalation of the lung lobes.

FIG. 2 is a perspective view of the bronchi emphasizing the upper right lung lobe 56. In addition to the bronchial branches illustrated in FIG. 1, FIG. 2 illustrates subsegmental bronchial branches 80, 82, 84, 86, 88, and 89 (fourth generation) providing air circulation to superior right lung lobe 56. The fifth- and sixth-generation bronchial branches are illustrated, but not given reference numbers.

The air passageways branch out, much like the roots of a tree. The bronchial segments branch into six generations or orders, and the bronchioles branch into approximately another three to eight generations or orders. Typically, each generation has a smaller diameter than its predecessor. The inside diameter of a generation varies depending on the particular bronchial branch, and further varies between individuals. For example, a typical lobar bronchus 42 (third generation) providing air circulation to the upper right upper lobe 56 has an internal diameter of approximately 1 cm. A typical segmental bronchi 48 (fourth generation) has an internal diameter of approximately 4 to 7 mm. The fifth and sixth generations (no reference numbers) are each proportionately smaller. The bronchial segments include annular ligaments and irregularly located cartilages that provide structure and resilience. The cartilages become increasingly sparse as the bronchial segments become smaller in diameter. The bronchioles do not have ligaments and cartilages.

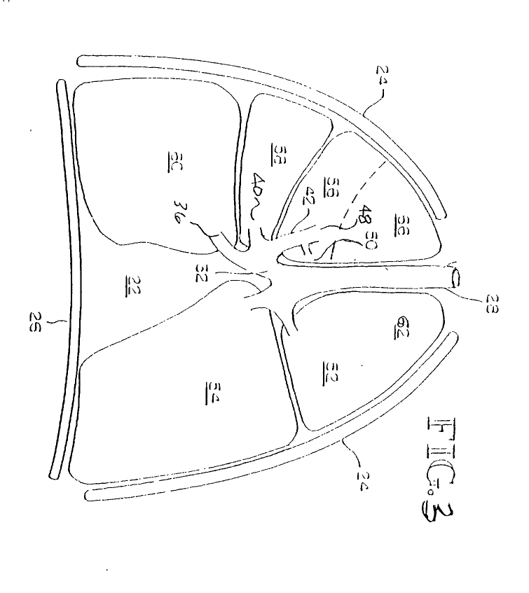

FIG. 3 illustrates a respiratory system suffering from COPD. In contrast to the lobes of FIG. 1, here it may be seen that the lung lobes 52, 54, 56, 58, and 60 are enlarged and that the diaphragm 26 is not arched but substantially straight. Hence, this individual is incapable of breathing normally by moving diaphragm 26. Instead, in order to create the negative pressure in thorax 22 required for breathing, this individual must move the chest wall outwardly to increase the volume of the thorax. This results in inefficient breathing causing these individuals to breathe rapidly with shallow breaths.

It has been found that the apex or segmental portions 62 and 66 of the upper lung lobes 52 and 56, respectively, are most affected by COPD. Hence, bronchial sub-branch obstructing devices are generally employed for treating the apex 66 of the right, upper lung lobe 56. The insertion of an obstructing member or a plurality of obstructing members treats COPD by deriving the benefits of lung volume reduction surgery without the need of performing the surgery. The intra-bronchial obstructions may be anchored in the air passageway to prevent movement or expulsion. In addition to treating COPD, it is presently contemplated that the intra-bronchial obstructions will be used for other purposes, including delivery of therapeutic substances.

The COPD treatment contemplates permanent collapse of a lung portion using at least one intra-bronchial obstruction. The collapse leaves extra volume within the thorax for the diaphragm to assume its arched state for acting upon the remaining healthier lung tissue. This should result in improved pulmonary function due to enhanced elastic recoil, correction of ventilation/perfusion mismatch, improved efficiency of respiratory musculature, and improved right ventricle filling. The treatment of COPD may include several intra-bronchial obstructing members being inserted in air passageways to form a redundant array. For example, if the volume of apex 66 of the right, upper lung lobe 56 were to be reduced, obstructing devices may be deployed in the four, fifth-generation air passageways branching off of the fourth-generation bronchial branches 80 and 82, redundant obstructing members placed in the fourth-generation bronchial branches 80 and 82, and another redundant obstructing member placed in the third-generation branch 50.

The physical characteristics of the obstructing devices currently available limit the range of air passageway diameters that a particular device can obstruct. The limiting characteristics include both the range of air passageway diameters that a single device can obstruct, and the range of air passageway diameters that can be engaged by anchors of the obstructing device. Use of anchors can allow the obstructing member to be relatively loosely fitted against the air passageway wall, which may preserve mucociliary transport of mucus and debris out of the collapsed lung portion. Thus, obstructing devices are provided in a variety of sizes for the various sizes of air passageways.

The present invention supports the use of intra-bronchial obstructing devices by enabling the inside diameter of the air passageway to be measured so that an appropriately sized obstructing device may be selected. As will be appreciated by those skilled the in art, the present invention may be used in conjunction with placing any type of obstructing member in an air passageway, including a plug, or a member that allows air passage in one direction but not another.

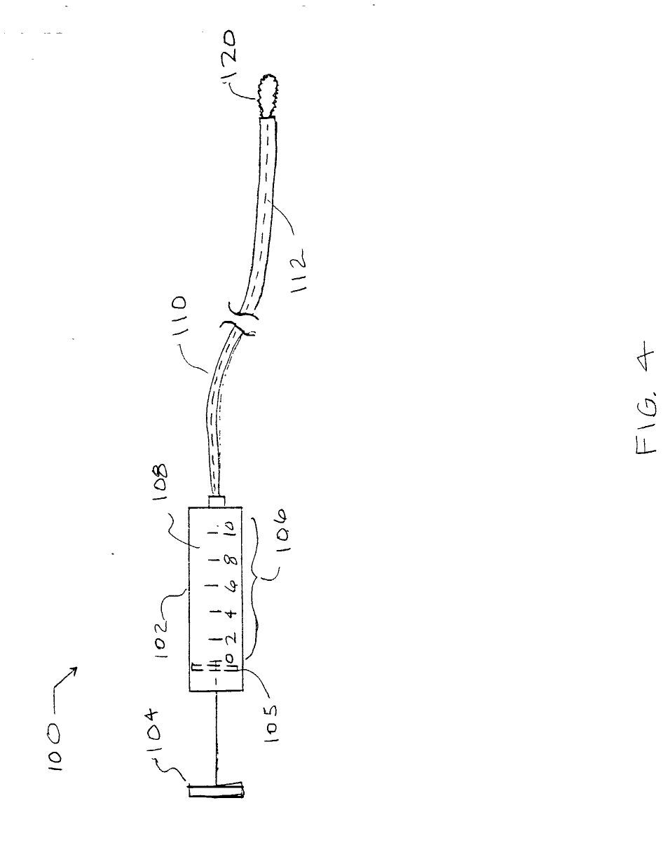

FIG. 4 illustrates an air passageway inside diameter measuring device 100 in accordance with the present invention. Measuring device 100 includes a fluid dispenser 102, a fluid 108, a flexible catheter 110, and a balloon 120.

The fluid dispenser (illustrated as a syringe 102) may be any device known in the art suitable for ejecting a measurable volume of the fluid 108 in amounts necessary to fill the balloon 120. The syringe 102 includes visually readable gradations 106 that correspond to air passageway diameters, and is illustrated with air passageway diameters ranging between zero and 10 mm. The gradations 106 may reflect any range of anticipated air passageway diameters. The syringe 102 also includes a handle 104 connected to a piston 105, and arranged such that moving the handle 104 transmits the motion to the piston 105, and the motion is further transmitted to the fluid 108.

The flexible catheter 110 may be any flexible, steerable, elongated tubular member arranged for transoral or transnasal insertion into an air passageway, and may be made from any suitable material known in the art, such as polyethylene. The catheter 110 includes an inflation lumen 112 arranged to be in fluid communication with the syringe 102. The catheter 110 is also arranged to carry and be in fluid communication with the balloon 120. In an embodiment, catheter 110 has an external diameter of approximately 2 mm. The catheter 110 may include opaque markings visible under X-ray fluoroscopy, such as gold or stainless steel, or other markings visible under other visualization methods.

The balloon 120 may be carried on the distal end of the catheter 110, or proximate to the distal end of the catheter 110. The balloon 120 may be made of any thin, flexible non-complaint material known in the art, such as polyurethane, suitable for use in air passageways. A balloon made of non-compliant material requires only a relatively low pressure for expansion. A non-compliant material provides a measurable or determinable relationship between balloon volume and an expandable transverse dimension. Balloon 120 may have any transverse cross-sectional shape that can be expanded adjacent to opposing portions of an air passageway wall. For example, while balloon 120 is generally described herein as having a round, expanded cross-section with a generally uniform single diameter, the balloon 120 may be any shape having a transverse dimension that can be expanded to contact opposing portions of an interior wall of an air passageway. For example, the balloon 120 may be an ellipsoidal transverse cross-section having an expandable transverse dimension that is expandable adjacent to opposing portions of an interior wall of air passageway 80. For purposes of clarity, aspects of the invention are described herein using a balloon 120 that expands into a round cross-section having an expanded transverse dimension that is a diameter. However, as stated above, the invention is not so limited.

Furthermore, in FIG. 4 the balloon 120 is illustrated in its deflated state for insertion and movement within air passageways. In its deflated state, the balloon 120 is approximately 10 mm in length and 2 mm in diameter. In its expanded state, the balloon 120 should be capable of expanding to more than the anticipated cross-sectional area of the air passageway being measured. Typically, the air passageway inside diameters being measured do not exceed 10 mm in diameter, so the balloon would have an expanded maximum diameter of approximately 12 mm. Because the air passageway diameter changes noticeably over a short distance, both the deflated and inflated lengths of the balloon are minimized so that a measurement for a particular location is not affected by the distal narrowing or proximal widening.

The fluid 108 may be any fluid suitable for use within the human body, such as a saline solution. A gas may be used, but a fluid is preferred to provide a discernable pressure increase in the inflation lumen 112 when the balloon 120 contacts the interior periphery of the air passageway.

The gradations 106 may be marked on the syringe 102 because the non-compliant material used for the balloon 120 provides a known relationship between the volume of the balloon 120 and its diameter. The gradations 106 start at “0” and are calibrated to correspond to the diameter of the expanded balloon 120, and thus the air passageway. As the handle 104 is pushed from “0” gradation, a measurable volume of the fluid 108, reflected by the other gradations, is ejected from the syringe 102 and forced into the balloon 120 through fluid communication by the lumen 112 of catheter 110. Because the relationship between the volume and the diameter of the balloon 120 is known, the diameter of the expanded balloon 120 can be determined from the volume of the fluid 108 ejected from the syringe 102. Diameters corresponding to a volume of fluid 108 ejected into the balloon 120 are marked in millimeter gradations 106 on the syringe 102. The diameter is determined by observing the location of the syringe piston 105 with respect to the gradations 106.

Points of correspondence for a particular configuration of balloon may be established on a test bench. Balloon 120 is expanded in a series of openings with several known diameters, and a correlation is established between the expanded volumes of the test balloon 120 and the several known diameters. The syringe gradations 106 are established correlating the volume of the fluid 108 displaced by movement from the “0” gradation with the known diameter. Each individual measuring device 100 may have its gradations determined on a test bench. Alternatively, the physical dimensions of the syringe 102 and the balloon 120 may be standardized, allowing standardized gradation markings 106.

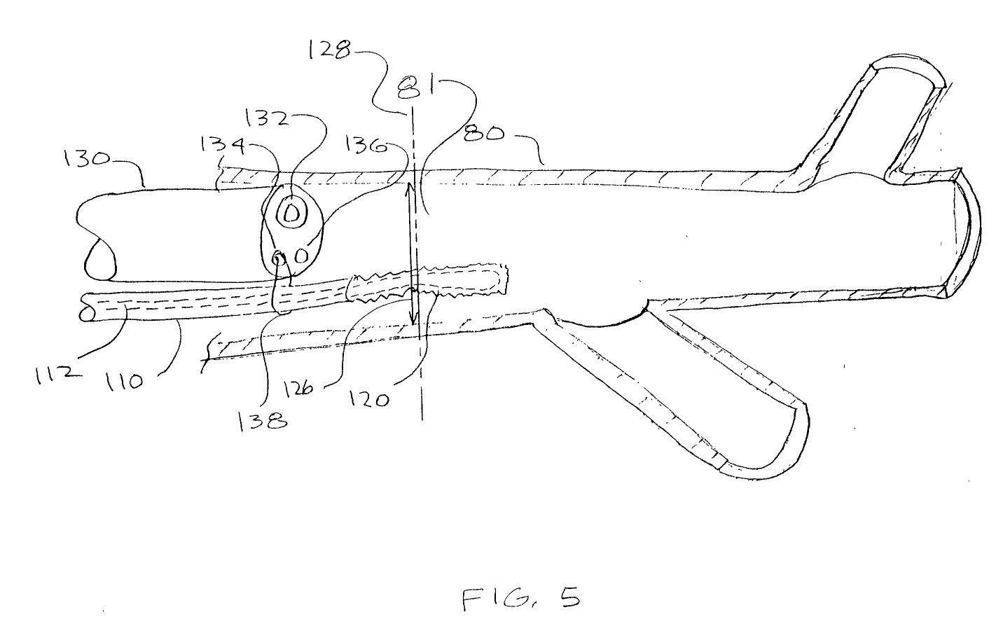

FIG. 5 illustrates a step in measuring an inside diameter 126 of an air passageway 81 at a measuring location 128 with the measuring device 100 of FIG. 4 in accordance with an aspect of the invention. In an embodiment of the measuring device 100, the syringe 102, the catheter 110, and the balloon 120 are provided already coupled together and ready for use. The syringe 102 is coupled to the lumen 112 at an end of the catheter 110. The balloon 120 is provided in its collapsed state and coupled to the lumen 112 at another end of the catheter 110. The syringe 102, the lumen 112, and the collapsed balloon 120 are filled with saline solution, the piston 105 is aligned with the “0” gradation of the gradations 106, and any air bubbles in the fluid have been removed. For clarity, inside diameter 126 is illustrated slightly displaced from measuring location 128. However, it is contemplated that measuring device 100 will measure the inside diameter 126 at the measuring location 128.

The distal end of catheter 110 and the balloon 120 may be transorally placed into the trachea 28 and steered into the air passageway 81 of the bronchus 80 to the measuring location 128 by any method and/or device known in the art. The catheter 110 may be steered into air passageway 81 by being carried in a working lumen 134 of a bronchoscope 130; associated with and then steered by the bronchoscope 130; inserted after the bronchoscope 130 is proximate to the measuring location 128 and steered adjacent to the shaft of the bronchoscope 130; or steered using imaging/visualization techniques, such as computed tomography or radiography.

FIG. 5 also illustrates an embodiment where the distal end of catheter 110 is associated with the bronchoscope 130 by cinching with a loop of material carried in the working lumen 134, such as dental floss 138. The distal end of the bronchoscope 130, with the associated distal end of the catheter 110 and the balloon 120, are steered into air passageway 81 for dimensioning. While the catheter 110 and the balloon 120 may be carried in a working lumen, it may be difficult to fully retract an expanded balloon 120 back into the working lumen for placement in another air passageway.

In another embodiment, the distal end of the bronchoscope 134 is steered into air passageway 81. Then the catheter 110 is steered alongside the bronchoscope 134 until it and balloon 120 can be observed in the viewing lens 132 of the bronchoscope 130.

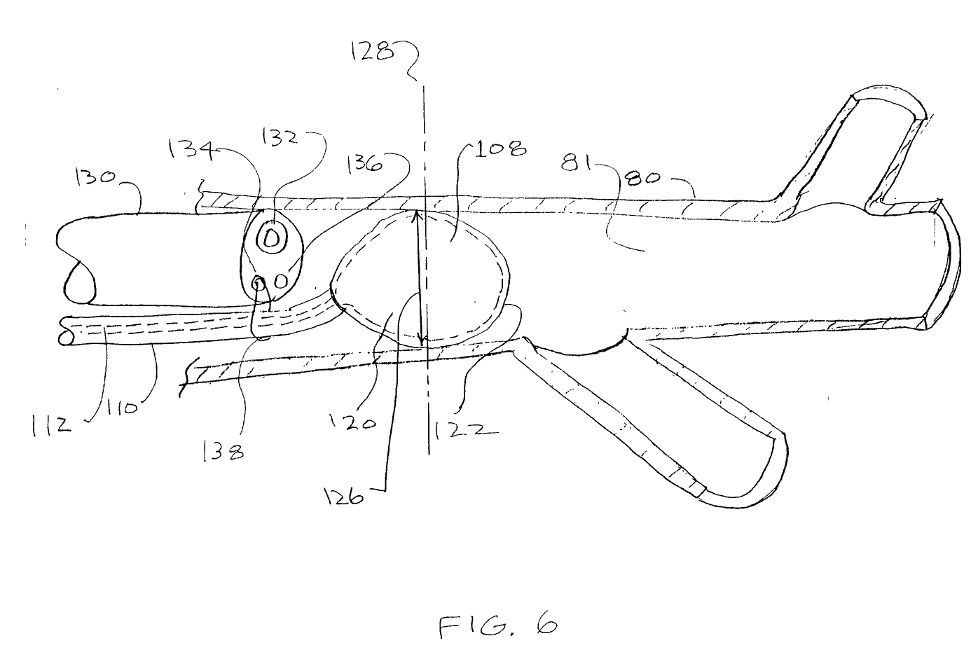

FIG. 6 illustrates another step in measuring an inside diameter 126 of an air passageway 81 at measuring location 128 with measuring device 100 of FIG. 4 in accordance with an aspect of the invention. Prior to expanding or inflating the balloon 120, the catheter 110 may be disassociated from the bronchoscope 130 by releasing one end of loop of dental floss 138 and pulling the other end of the dental floss from the working lumen 134. Also prior to expanding the balloon 120, the syringe piston 105 has been initially set at the “0” gradation of gradations 106, and all air bubbles have been removed from the fluid 108. The balloon 120 is expanded in the air passageway 81 by using the handle 104 of the syringe 102 to eject a portion of the fluid 108 into the inflation lumen 112 and correspondingly into the balloon 120.

Ejection of fluid 108 and expansion of the balloon 120 within the air passageway continues until the periphery 122 of the balloon 120 is at least adjacent to the inside wall of the air passageway 81 at measuring location 128. As used herein, “adjacent” means closing the space between the periphery 122 of the balloon 120 and an interior periphery of an interior wall of the air passageway 81 for confirmation by visual means, and means physically contacting an interior periphery for confirmation by pressure sensing means. Once the balloon 120 expands to a point where its periphery 122 at the measuring location 128 is adjacent to an interior periphery 81 of the air passageway 80, the expanded transverse dimension of the balloon 120 is the same as the inside diameter 126 of the air passageway 80. In the embodiment illustrated in FIG. 6, adjacency between the periphery 122 of the balloon 120 and the inside wall of the air passageway 81 at measuring location 128 is visually confirmed by observation through the viewing lens 132 of the bronchoscope 130. When adjacency exists, the diameter 126 at measuring location 128 is read by the alignment of the syringe piston 105 with one or more of the gradations 106. If the balloon 120 has an expanded transverse cross-section that is not round, the expandable transverse dimension is expanded to a point where a portion of its periphery 122 at the measuring location 128 is adjacent to opposing portions of the interior periphery 81 of the interior wall of the air passageway 80.

The structure and resilience of the bronchi resist expansion beyond the bronchi's natural or normal diameter. This resistance provides a discernable pressure increase, or a pressure spike, in the inflation lumen 112 of the balloon 120 when expansion beyond the normal or natural diameter is attempted. Because of the resistance to further expansion, contact may also be tactilely perceived by an increase in the force required to eject the fluid 108 from the syringe 102. In an alternative embodiment, a controller may be used to sense this resistance, and to prevent further ejection of fluid by the syringe, thus locking-in the diameter reading. The controller may be mechanical or electronic, or a combination.

After the measurement is taken, the measuring device 100 is arranged to allow the balloon 120 to be deflated by drawing the fluid 108 back into the syringe 102 while the balloon 120 is within the air passageway 81. The catheter 110 and the deflated balloon 120 may then be steered to another measuring location, and another air passageway diameter measured.

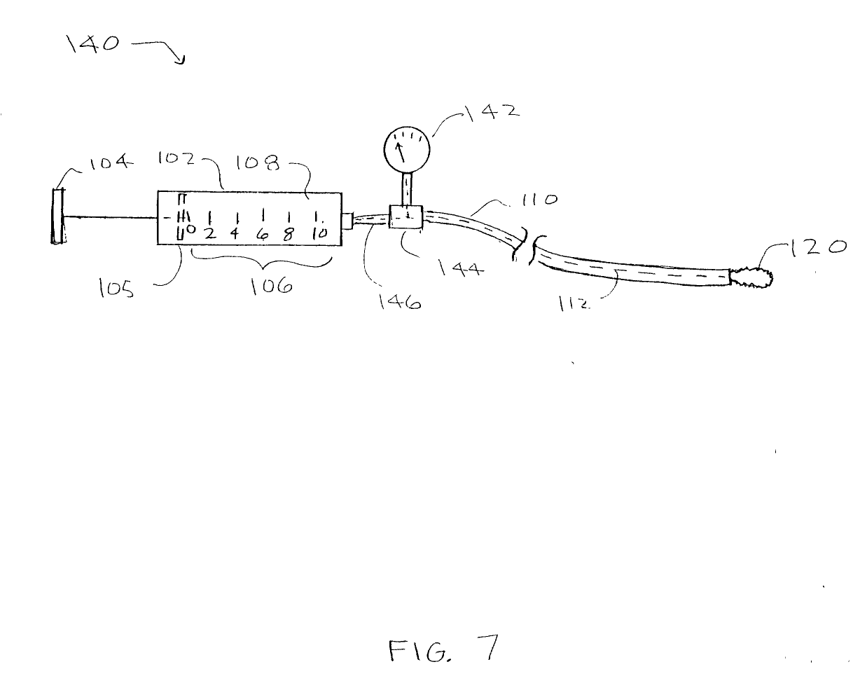

FIG. 7 illustrates another embodiment of an air passageway inside diameter measuring device 140 in accordance with the present invention. Measuring device 140 is similar to measuring device 100 of FIG. 4, and includes a port 144 coupled to inflation lumen 112 for sensing pressure within the lumen, and a pressure indicator 142. The port 144 may be incorporated in syringe 102, or optionally may be a separate component coupled to syringe 102 by a coupler 146. Pressure indicator 142 may be any device known to those in the art operable to sense and indicate the pressure of the fluid 108 in the inflation lumen 112. Pressure indicator 142 may be any type of device or combination of devices operable to sense and indicate pressure, including mechanical, electrical, or a combination thereof.

In operation, the catheter 110 and the balloon 120 of the measuring device 140 are placed in the air passageway 81 in the same manner as described for measuring device 100 in FIGS. 5 and 6. Instead of visually confirming when the expanded balloon 120 is adjacent to a wall of the air passageway 81, the pressure of the fluid 108 in the inflation lumen 112 is monitored. The inflation lumen 112 pressure during expansion of the balloon 120 should typically be relatively uniform and in the neighborhood of 300 mmHg. A pressure spike in the neighborhood of 500 mmHg should occur when the balloon 120 contacts the wall of the air passageway 81 and further expansion should be opposed by the structure of the bronchus 80. When the pressure in the inflation lumen equals a predetermined level, which is 500 mmHg for this embodiment, movement of the syringe handle 104 is terminated and the gradations 106 are read to determine the diameter of the air passageway 81.

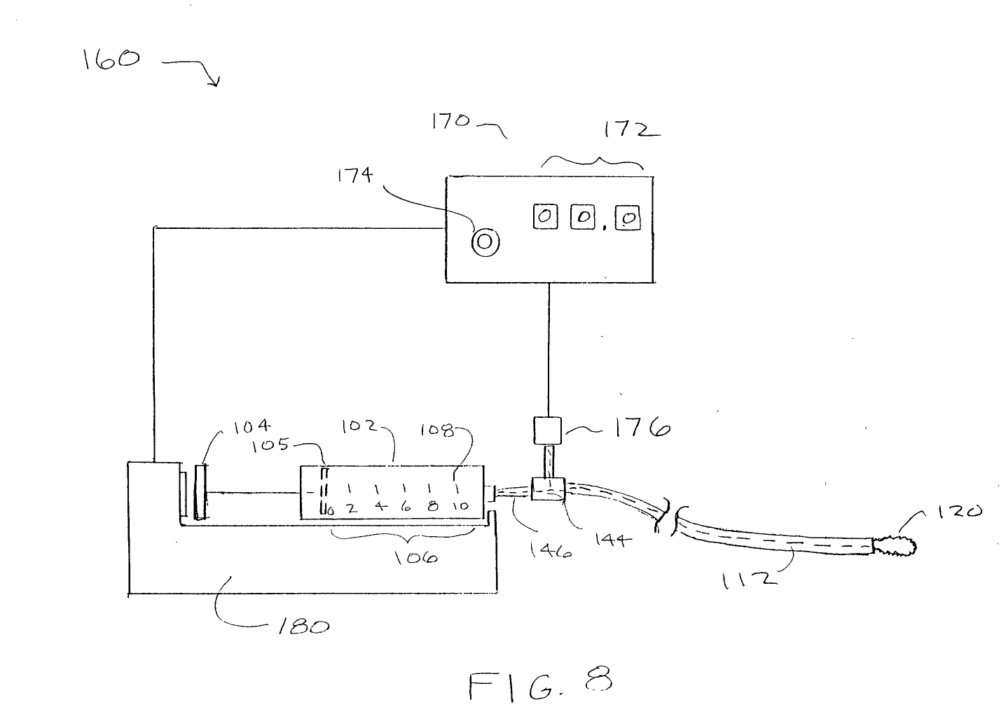

FIG. 8 illustrates another embodiment of an air passageway inside diameter measuring device 160 in accordance with the present invention. The measuring device 160 is similar to the measuring device 140, and additionally includes a controller 170 and an automatic fluid dispenser illustrated as a syringe pump 180. The controller 170 includes a digital display 172, an indicator light 174, and a pressure sensor 176.

The controller 170 is coupled to the syringe pump 180, and to the pressure sensor 176 that is coupled to the inflation lumen 112. Controller 170 is operable to control fluid ejection from the syringe pump 180, sense pressure in the inflation lumen 112, determine volume of fluid 108 ejected from the syringe 102 before a predetermined pressure occurs in the inflation lumen 112, correlate volume of fluid 108 ejected to diameter of the balloon 120, determine air passageway diameter in response to volume of fluid 108 ejected, and display determined air passageway diameter on the digital display 172. Controller 170 may also be operable to activate the indicator display 174, and optionally to activate an audible indicator (not shown) when pressure in the inflation lumen 112 exceeds a predetermined level. Controller 170 may be any device, including electrical, mechanical, or a combination thereof, and may include a computing device, an ASIC, and/or a microprocessor.

Syringe pump 180 may be any device known in the art, including electrical, mechanical, or a combination thereof, arranged to eject a measurable volume of the fluid 108, which may be from a syringe such as the syringe 102, in response to controller 170. Sensor 176 may be any device known in the art, including electrical, mechanical, or a combination thereof, arranged to provide a signal to controller 170 in response to the pressure in inflation lumen 112. Digital display 172 may be any device known in the art, including an LCD, a series of LEDs, or an electrical or mechanical device, or a combination thereof, arranged to provide a numerical display representing an air passageway diameter. Indicator light 174 may be any device known in the art, including an LED, arranged to illuminate in response a signal from controller 170.

In operation, the catheter 110 and balloon 120 of measuring device 160 are placed in the air passageway 81 in the same manner as described for measuring device 100 in FIGS. 5 and 6. The controller 170 activates the syringe pump 180, and controls the ejection of a volume of the fluid 108 from the syringe 102 into inflation lumen 112. The balloon 120 expands in response to the ejected fluid 108, and the sensor 176 senses pressure in the inflation lumen 112 and provides a signal to the controller 170. Instead of visually confirming when the expanded balloon 120 contacts the wall of the air passageway 81, the pressure of the fluid 108 in the inflation lumen 112 is monitored by controller 170. When the balloon 120 contacts the wall of the air passageway 81 and further expansion is opposed by the structure of the air passageway 80, a predetermined pressure occurs in the lumen 112 that is sensed by the sensor 176, which provides a signal to the controller 170. The controller 170 stops ejection of the fluid 108, determines the volume of the fluid 108 ejected from the syringe 102, correlates the volume to the diameter of the balloon 120 according to a look-up table or other data stored in the controller 170 to determine the diameter of the balloon 120, and displays the diameter of the balloon 120 as the diameter of the air passageway 81 on the display 172. Optionally, the controller 170 also activates indicator light 174 and the audible device (not shown) when the predetermined pressure occurs in the lumen 112.

Although the present invention has been described in detail with reference to certain preferred embodiments, other embodiments are possible. Therefore, the spirit or scope of the appended claims should not be limited to the description of the embodiments contained herein. It is intended that the invention resides in the claims hereinafter appended.

What is claimed is:

1. A device for measuring an inside diameter of an air passageway, the device comprising:
a flexible catheter having an inflation lumen;
a fluid dispenser in fluid communication with the inflation lumen and operable to eject a measurable volume of fluid; and
a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension.

2. The device of claim 1, wherein the balloon member is dimensioned for transoral placement into the air passageway.

3. The device of claim 1, wherein the catheter is configured to be steerable within bronchi.

4. The device of claim 1, wherein the balloon member comprises a non-complaint material.

5. The device of claim 1, wherein the balloon member is arranged to expand into contact with the air passageway wall.

6. The device of claim 1, wherein the transverse dimension of the balloon is arranged to expand to a dimension of between 3 mm and 12 mm.

7. The device of claim 1, wherein the balloon has a deflated configuration for placement in the air passageway and an inflated configuration for measuring a diameter of the air passageway.

8. The device of claim 7, wherein the balloon is arranged to transition from the inflated configuration to the deflated configuration while in the air passageway, and then to transition from the deflated configuration to a re-inflated configuration for measuring the diameter of another air passageway.

9. The device of claim 1, wherein the fluid dispenser comprises a syringe.

10. The device of claim 1, wherein the fluid dispenser comprises a syringe pump.

11. The device of claim 1, wherein the fluid dispenser further comprises gradations corresponding to air passageway diameters.

12. The device of claim 1, wherein the catheter has a distal end, and the balloon is carried on the catheter proximate to the distal end of the catheter.

13. An assembly for use in measuring the inside diameter of an air passageway, the assembly comprising:
a flexible catheter having an inflation lumen, the inflation lumen being arranged for fluid coupling with a fluid dispenser operable to eject a measurable volume of fluid; and
a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension.

14. The assembly of claim 13, wherein the balloon is carried on the catheter.

15. The assembly of claim 14, wherein the catheter has a distal end, and the balloon is carried proximate to the distal end of the catheter.

16. The device of claim 13, wherein the balloon member is arranged to expand into contact with the air passageway wall.

17. A device for measuring an inside diameter of an air passageway, the device comprising:
a flexible catheter having an inflation lumen;
a fluid dispenser in fluid communication with the inflation lumen and operable to eject a measurable volume of fluid;
a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension; and
a port in fluid communication with the inflation lumen allowing sensing of pressure in the inflation lumen.

18. The device of claim 17, wherein the device further comprises a pressure indicator, and the pressure indicator is coupled to the port.

19. The device of claim 18, wherein the pressure indicator is arranged to measure luminal pressure between zero mmHg and 700 mmHg.

20. The device of claim 17, wherein the pressure indicator comprises a pressure sensor that generates a sensor signal and an indicator operable to indicate luminal pressure in response to the sensor signal.

21. The device of claim 17, wherein the fluid dispenser comprises a syringe.

22. The device of claim 17, wherein the fluid dispenser comprises a syringe pump.

23. The device of claim 17, wherein the device further comprises:
a pressure sensor coupled to the port; and
a controller coupled to the fluid dispenser and the pressure sensor, the controller being operable to control fluid ejection, determine volume of fluid ejected before a predetermined pressure occurs in the inflation lumen, determine air passageway diameter in response to volume of fluid ejected, and display determined air passageway diameter.

24. The device of claim 17, wherein the balloon member is arranged for transoral placement into the air passageway.

25. The device of claim 17, wherein the device further comprises:
a pressure sensor coupled to the port; and
a controller coupled to the fluid dispenser and the pressure sensor, the controller being operable to stop fluid ejection into the balloon member when a predetermined pressure occurs in the inflation lumen.

26. A device for measuring an inside diameter of an air passageway, the device comprising:
a flexible catheter having an inflation lumen;
a fluid dispenser in fluid communication with the inflation lumen and operable to eject a measurable volume of fluid;
a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension; and
a pressure indicator coupled to the inflation lumen for indicating pressure in the inflation lumen.

27. The device of claim 26, wherein the balloon member is arranged to expand into contact with the air passageway wall.

28. A device for measuring the inside diameter of a body lumen, the device comprising:
a flexible catheter having an inflation conduit;
a fluid dispenser in fluid communication with the inflation conduit and operable to eject a measurable volume of fluid; and
a balloon member in fluid communication with the inflation lumen and expandable to a transverse dimension adjacent to opposing portions of an interior wall of the air passageway in response to fluid ejected from the fluid dispenser, and having a known relationship between volume and the expandable transverse dimension.

29. A method of measuring an air passageway inside diameter, the method including the steps of:
placing a balloon in the air passageway, the balloon being expandable to a transverse dimension adjacent to opposing portions of an interior wall of the air passageway and having a known relationship between volume and the expandable transverse dimension;
expanding the balloon until the expandable transverse dimension is adjacent to opposing portions of an inner periphery of the air passageway; and
determining the air passageway diameter in response to the volume of the expanded balloon.

30. The method of claim 29, further including the step of detecting adjacency to the inner periphery of the air passageway.

31. The method of claim 30, wherein the step of detecting adjacency includes the further step of visually establishing adjacency.

32. The method of claim 29, wherein the step of expanding the balloon includes the further step of expanding the balloon until it contacts the opposing portions, and the method further includes the step of detecting contact by sensing a predetermined pressure in the balloon.

33. The method of claim 29, further including the step of placing a fluid dispenser in fluid communication with the balloon, the fluid dispenser being operable to inject a measurable volume of fluid into the balloon, and the step of expanding the balloon includes the further step of injecting a measurable volume of fluid into the balloon.

34. The method of claim 33, wherein the fluid dispenser comprises a syringe.

35. The method of claim 33, wherein the fluid dispenser comprises gradations related to air passageway diameter, and the step of determining air passageway diameter includes the further step of observing the gradations.

36. The method of claim 29, wherein the step of placing a balloon in the air passageway includes the further step of transorally placing the balloon in the air passageway.

37. A device for measuring an inside diameter of an air passageway, the device comprising:
means for placing a balloon member in the air passageway, the balloon member expandable in a transverse dimension adjacent to opposing portions of an interior wall of the air passageway and having a known relationship between volume and expandable transverse dimension;
means for expanding the balloon until the expandable transverse dimension is adjacent to opposing portions of an inner periphery of the air passageway; and
means for determining the air passageway diameter in response to the volume of the expanded balloon.
